(19) United States
(12) Patent Application Publication
(10) Pub. No.: US 2008/0183265 A1
(43) Pub. Date: Jul. 31, 2008

(54) TRANSVASCULAR LEAD WITH PROXIMAL FORCE RELIEF

(75) Inventors: Mark J. Bly, Falcon Heights, MN (US); Randy W. Westlund, River Falls, WI (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/669,039

(22) Filed: Jan. 30, 2007

(51) Int. Cl. A61N 1/05

(52) U.S. Cl. 607/122

(57) ABSTRACT

A medical electrical lead for implantation in a patient's internal jugular vein at a target location and adjacent a vagus nerve. The lead comprises a proximal region having a proximal stiffness and a distal region. The distal region has a distal stiffness and a first spiral configured to retain the distal region in the internal jugular vein. A transition region is interposed between the proximal and distal regions and has a transitional stiffness. An electrode is coupled to the distal region. The proximal stiffness is less than the distal stiffness so as to reduce an amount of force transferred from the proximal region to the distal region. The transitional stiffness is less than the distal stiffness and greater than the proximal stiffness.

### CROSS-REFERENCE TO RELATED APPLICATIONS

This application is related to the following co-pending and co-owned applications: DUAL SPIRAL LEAD CONFIGURATIONS, filed on the same day and assigned Ser. No. ______; ELECTRODE CONFIGURATIONS FOR TRANSVASCULAR NERVE STIMULATION, filed on the same day and assigned Ser. No. ______; SPIRAL CONFIGURATIONS FOR INTRAVASCULAR LEAD STABILITY, filed on the same day and assigned Ser. No. ______; METHOD AND APPARATUS FOR DELIVERING A TRANSVASCULAR LEAD, filed on the same day and assigned Ser. No. ______; NEUROSTIMULATING LEAD HAVING A STENT-LIKE ANCHOR, filed on the same day and assigned Ser. No. ______; METHOD AND APPARATUS FOR DIRECT DELIVERY OF TRANSVASCULAR LEAD, filed on the same day and assigned Ser. No. ______; and SIDE PORT LEAD DELIVERY SYSTEM, filed on the same day and assigned Ser. No. ______, all herein incorporated by reference in their entirety.

### TECHNICAL FIELD

The present invention relates to medical electrical leads for nerve or muscle stimulation. The present invention more particularly relates to medical electrical leads having improved retention in an internal jugular vein.

### BACKGROUND

A significant amount of research has been directed both to the direct and indirect stimulation of nerves including the left and right vagus nerves, the sympathetic and parasympathetic nerves, the phrenic nerve, the sacral nerve, and the cavernous nerve to treat a wide variety of medical, psychiatric, and neurological disorders or conditions. More recently, stimulation of the vagus nerve has been proposed as a method for treating various heart conditions, including heart failure. Heart failure is a cardiac condition characterized by a deficiency in the ability of the heart to pump blood throughout the body and high filling pressure causing pulmonary fluid to build up in the lungs.

Typically, nerve stimulating electrodes are cuff- or impalement-type electrodes placed in direct contact with the nerve to be stimulated. These electrodes require surgical implantation and can cause irreversible nerve damage due to swelling or direct mechanical damage to the nerve. A less invasive approach is to stimulate the nerve through an adjacent vessel using an intravascular lead. A lead including one or more electrodes is inserted into a patient's vasculature and delivered to a site within a vessel adjacent a nerve to be stimulated.

Retaining a lead inside of a vessel for intravascular nerve stimulation presents difficulties. For example, the diameter and cross-section of a patient's internal jugular vein can vary depending upon whether the patient is lying down or standing up. Also, movement of the neck and external pressure on the neck can dislodge a lead located inside the internal jugular vein. Thus, there is a need in the art for a medical electrical lead that can be reliably retained in the internal jugular vein.

### SUMMARY

In one embodiment, the invention is a medical electrical lead for implantation in a patient's internal jugular vein at a target location and adjacent a vagus nerve. The lead comprises a proximal region having a proximal stiffness and a distal region. The distal region has a distal stiffness and a first spiral configured to retain the distal region in the internal jugular vein. A transition region is interposed between the proximal and distal regions and has a transitional stiffness. An electrode is coupled to the distal region. The proximal stiffness is less than the distal stiffness so as to reduce an amount of force transferred from the proximal region to the distal region. The transitional stiffness is less than the distal stiffness and greater than the proximal stiffness.

In another embodiment, the invention is a medical electrical lead for implantation in a patient's internal jugular vein at a target location and adjacent a vagus nerve. The lead comprises a proximal region, a distal region, and an electrode coupled to the distal region. The proximal region includes means for reducing an amount of force transferred from the proximal region to the distal region. The distal region includes means for retaining the distal region in the internal jugular vein.

In another embodiment, the invention is a medical electrical lead for implantation in a patient's internal jugular vein at a target location and adjacent a vagus nerve. The lead comprises a proximal region having a proximal stiffness and a distal region. The distal region has a distal stiffness and a retaining structure configured to retain the distal region in the internal jugular vein. An electrode is coupled to the distal region. The proximal stiffness is less than the distal stiffness so as to reduce an amount of force transferred from the proximal region to the distal region.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a schematic view of a patient's upper torso.

FIG. 2 shows a schematic view of a medical electrical lead implanted in an internal jugular vein according to one embodiment of the present invention.

FIG. 3 shows a schematic view of the medical electrical lead of FIG. 2.

FIG. 4 shows a front view of a medical electrical lead according to another embodiment of the present invention.

FIG. 5 shows a front view of a medical electrical lead according to another embodiment of the present invention.

FIG. 6 shows a schematic view of a medical electrical lead implanted in an internal jugular vein according to yet another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

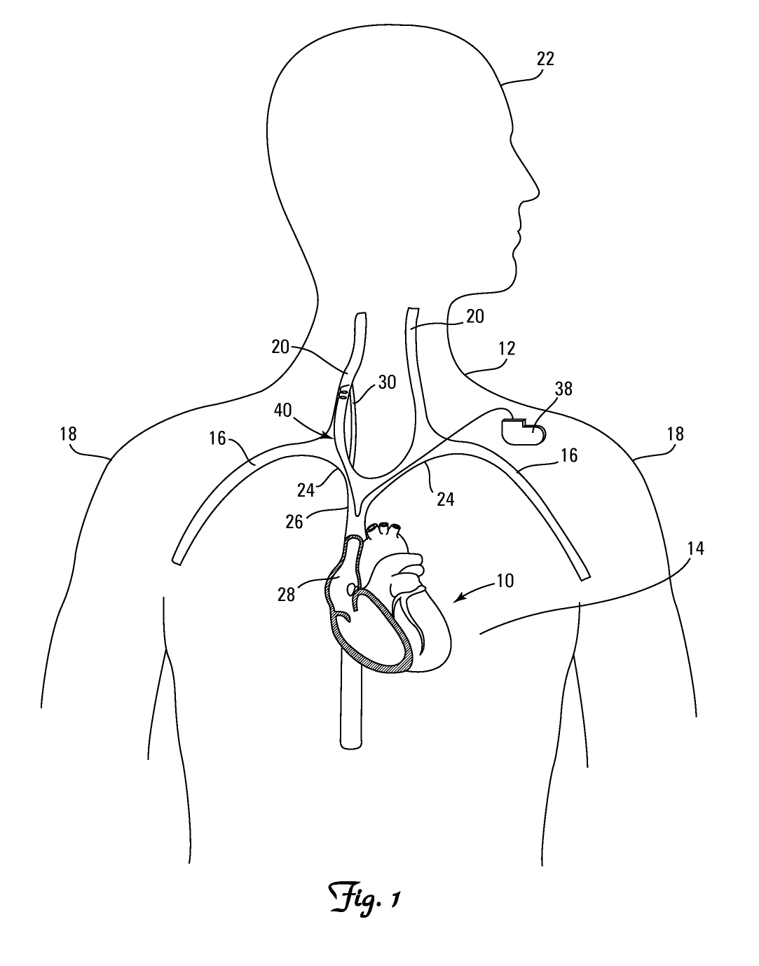

FIG. 1 shows a schematic view of a patient's upper torso, including a heart 10 and the veins of the neck 12 and thorax 14. The subclavian veins 16 drain blood from the arms 18. The internal jugular veins 20 drain blood from the head 22 and join the subclavian veins 16 to form the brachiocephalic or innominate veins 24. The union of the brachiocephalic veins 24 forms the superior vena cava 26, which returns blood from the head 22, neck 12, arms 18, and thorax 14 to the right atrium 28. A vagus nerve 30 is shown adjacent to the right internal jugular vein 20. Another vagus nerve (not shown) is adjacent to the left internal jugular vein 20. A stimulating device 38 is located in a subcutaneous pocket near the patient's subclavian vein 16. The stimulating device 38 is connected to a medical electrical lead 40 extending through the patient's subclavian, brachiocephalic, and internal jugular veins 16, 24, 20. In one embodiment, the stimulating device 38 provides electrical stimulation to the vagus nerve 30.

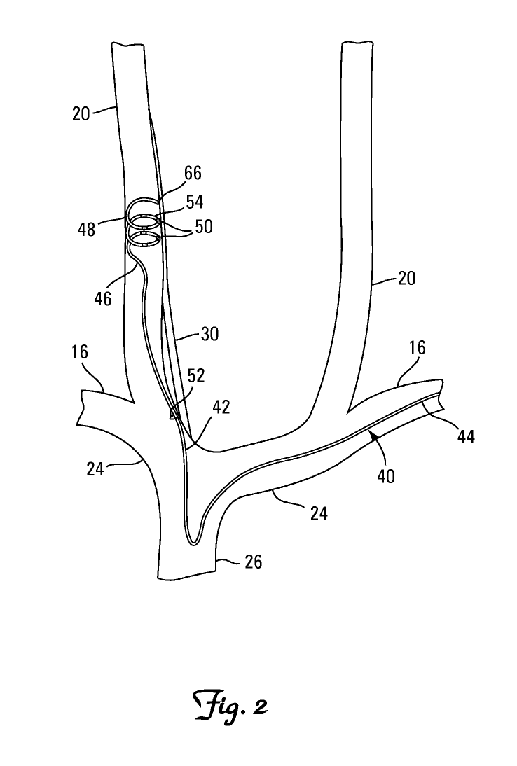

FIG. 2 shows a schematic view of a medical electrical lead 40 extending through a patient's subclavian vein 16 and brachiocephalic vein 24 and implanted in the patient's internal jugular vein 20 according to one embodiment of the present invention. Although the medical electrical lead 40 in FIG. 2 is shown implanted using an “opposite side method” from the left subclavian vein 16 into the right internal jugular vein 20, in another embodiment, the medical electrical lead 40 is implanted from the right subclavian vein 16 into the left internal jugular vein 20. In yet another embodiment, the implantation of the medical electrical lead 40 is a “same side” implantation from the right subclavian vein 16 into the right internal jugular vein 20, or the left subclavian vein 16 into the left internal jugular vein 20.

As illustrated in FIG. 2, the medical electrical lead 40 includes a lead body 42 comprised of an electrically insulative material. The medical electrical lead 40 includes a proximal region 44 and a distal region 46. A retaining structure 48 is located in the distal region 46. Electrodes 50 are located in the distal region 46 and are electrically coupled to the stimulating device 38 through conductive members (not shown). Although two electrodes 50 are shown in FIG. 2, the medical electrical lead 40 can include any number of electrodes 50. The electrodes 50 can comprise ring electrodes or can have any other configuration as is known in the art. In another embodiment, the electrodes 50 are configured according to related and commonly assigned U.S. patent application Ser. No. ______, filed ______, 2007, entitled ELECTRODE CONFIGURATIONS FOR TRANSVASCULAR NERVE STIMULATION, above-incorporated by reference in its entirety. The distal region 46 of the medical electrical lead 40 has a stiffness that is greater than the stiffness of the proximal region 44. The stiffness of the distal region 46 and the retaining structure 48 exert a force against the internal jugular vein 20. This force aids in retaining the electrodes 50 against the internal jugular vein 20 and adjacent to the vagus nerve 30, as well as in stabilizing the lead 40 within the internal jugular vein 20.

Improved retention of the distal region 46 is advantageous in the context of implanting a medical electrical lead 40 in the internal jugular vein 20 due to variability of the diameter and cross-section of a patient's internal jugular vein depending on the patient's position and the effect of movement of and external pressure on the patient's neck 12. Thus, the increased stability of the medical electrical lead 40 improves its ability to reliably deliver chronic therapy. The lower stiffness of the proximal region 44 reduces the amount of force transferred from the proximal region 44 to the distal region 46. Additionally, the lower stiffness of the proximal region 44 allows the implanting clinician to add lead slack into the superior vena cava 26 (as shown in FIG. 1) when implanting the medical electrical lead 40 into the internal jugular vein 20. The lead slack also aids in retaining the distal region 46 in the internal jugular vein 20.

The stiffness of the various regions of the lead 40 may be measured using any standard method. One method includes measuring the force required to bend or deflect a 10 mm section of the lead 40 a distance of 0.5 mm. Using this method, a section of the lead 40 is cut to a distance greater than 10 mm, for example, 15 mm. The 15 mm section of the lead 40 is then secured at two points with a distance of 10 mm between the two points. A force is applied to the center of the two points and the distance of deflection at various amounts of force measured. The amount of force required to deflect the 10 mm section a distance of 0.5 mm may be used as the measurement of the stiffness of the lead 40. The force may be measured in miliNewtons (mN). The stiffness of a lead 40 that is less than 10 mm is measured by forming an elongated section of the lead 40 that is greater than 10 mm and contains the same components as the section of the actual lead 40.

In one embodiment, the stiffness of the proximal region 44 is such that the force required for deflection of 0.5 mm over a 10 mm span is less than approximately 500 mN. In another embodiment, the stiffness of the proximal region 44 is such that the force required for deflection of 0.5 mm over a 10 mm span is less than approximately 300 mN. The relative stiffness of the distal region 46 to the proximal region 44 can be described in terms of a ratio. In one embodiment, the ratio of the stiffness of the distal region 46 to the proximal region 44 is approximately 2:1. In another embodiment, the ratio of the stiffness of the distal region 46 to the proximal region 44 is approximately 4:1. In another embodiment, the ratio of the stiffness of the distal region 46 to the proximal region 44 is any ratio that permits retention of the distal region 46 in the internal jugular vein 20 and reduces the amount of force transferred from the proximal region 44 to the distal region 46.

The medical electrical lead 40 can be further stabilized in the internal jugular vein 20 by using a suture 52 in the distal region 46. In one embodiment, the medical electrical lead 40 is further stabilized through the wearing of a neck brace by the patient for a period of time after implantation of the medical electrical lead 40. In an alternative embodiment, the medical electrical lead 40 can include fixation features well known in the art, such as silicone tines or a corkscrew-shaped fixation feature (not shown) at the distal region 46, to stabilize the medical electrical lead 40 in the internal jugular vein 20. In an alternative embodiment, the fixation feature can be located on the retaining structure 48. In other embodiments, the fixation feature can be located at a tip 66 of the medical electrical lead 40. The medical electrical lead 40 can also include an area 54 on the lead body 42 that promotes tissue in-growth. In one embodiment, the area 54 includes a roughened polymer surface on the lead body 42. In alternative embodiments, the area 54 includes a region of stepped or inset diameter within the lead body 42, within an electrode 50, or between the lead body 42 and an electrode 50. In other embodiments, the area 54 includes a polymer mesh, for example, a Dacron mesh, a metal mesh, for example, a stainless steel or nitinol mesh, or a bio-absorbable mesh. Examples of a bio-absorbable mesh include polyglycolic acide, poly-lactic acid, and polydioxanone. The medical electrical lead 40 can include any combination of sutures 52, fixation devices, tissue in-growth areas 54, or a neck brace to improve its stability within the internal jugular vein 20.

The medical electrical lead 40 can be implanted in the internal jugular vein 20 or any other vessel using a percutaneous stick method. A stylet or guidewire (not shown) can be used to implant the medical electrical lead 40 in the vessel. In one embodiment, a stylet or guidewire can be used to impart increased stiffness to the proximal region 44 during the implant procedure. In alternative embodiments, the medical electrical lead 40 can be implanted using a lead delivery system such as those disclosed in related and commonly assigned U.S. patent application Ser. No. ______, filed ______, 2007, entitled METHOD AND APPARATUS FOR DIRECT DELIVERY OF TRANSVASCULAR LEAD, related and commonly assigned U.S. patent application Ser. No. ______, filed ______, 2007, entitled METHOD AND APPARATUS FOR DELIVERING TRANSVASCULAR LEAD, and related and commonly assigned U.S. patent application Ser. No. ______, filed ______, 2007, entitled SIDE PORT LEAD DELIVERY SYSTEM, all above-incorporated by reference in their entirety.

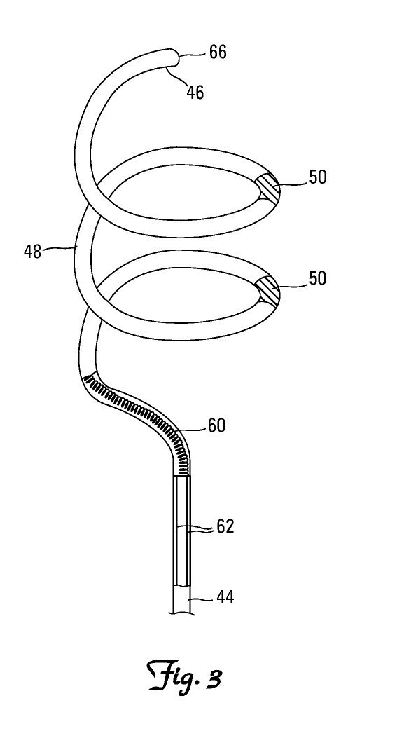

FIG. 3 shows a combined schematic view of the medical electrical lead 40 of FIG. 2. As illustrated in FIG. 3, the retaining structure 48 comprises a spiral. In one embodiment, the retaining structure 48 has a spiral shape as disclosed in related and commonly assigned U.S. patent application Ser. No. ______, filed ______, 2006, entitled SPIRAL CONFIGURATIONS FOR INTRAVASCULAR LEAD STABILITY, above-incorporated by reference in its entirety. In an alternative embodiment, the retaining structure 48 has the form of a bifurcated, bidirectional, or double spiral as disclosed in related and commonly assigned U.S. patent application Ser. No. ______, filed ______, 2007, entitled DUAL SPIRAL LEAD CONFIGURATIONS, above-incorporated by reference in its entirety. In other embodiments, the retaining structure 48 has any shape that retains the electrode 50 at a desired location within the internal jugular vein 20. In one embodiment, the retaining structure 48 has a diameter between approximately 5 and approximately 50 percent greater than the inner diameter of the jugular vein 20. In one embodiment, the retaining structure 48 has a diameter that is approximately 2 millimeters greater than the internal diameter of the internal jugular vein 20.

As discussed with respect to FIG. 2, the distal region 46 of the medical electrical lead 40 has a stiffness that is greater than the stiffness of the proximal region 44. In the embodiment shown in FIG. 3, the distal region 46 includes a conductive member 60 and the proximal region 44 includes a conductive member 62. The greater stiffness of the distal region 46 relative to the stiffness of the proximal region 44 results from the greater stiffness of the conductive member 60 relative to the conductive member 62. In the embodiment illustrated in FIG. 3, the conductive member 60 comprises a conductive coil and the conductive member 62 comprises a cable connector having a stiffness less than the stiffness of the conductive coil 60. In an alternative embodiment, both conductive members 60, 62 comprise conductive coils, but the conductive coil 60 has a greater stiffness than the conductive coil 62. This increased stiffness can be accomplished through differences in winding the coils 60, 62, selecting different materials for the coils 60, 62, differences in the filar diameter for the coils 60, 62, differences in the pitch of the coils 60, 62, or using any other method as is known in the art. In one embodiment, the conductive member 60 is made of a stiffer metal than the conductive member 62. In one embodiment, the coil 60 is made of MP35N—Ag and the conductive member 62 is made of Pt—Ta. In one embodiment, the conductive member 60 could be a conductive cable that is stiffer than the coil conductive member 62.

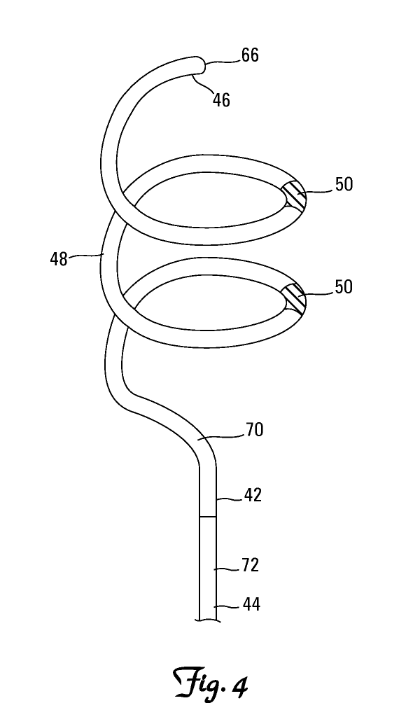

FIG. 4 shows a front view of a medical electrical lead 40 according to another embodiment of the present invention. In this embodiment, the difference in stiffness between the distal region 46 and the proximal region 44 is accomplished through the selection of different lead body materials. The lead body 42 has a distal lead body 70 and a proximal lead body 72. In one embodiment, the distal lead body 70 is comprised of a stiffer polymer and the proximal lead body 72 is comprised of a more flexible polymer. In one embodiment, the distal lead body 70 comprises polyurethane and the proximal lead body 72 comprises silicone. In an alternative embodiment, the distal lead body 70 and the proximal lead body 72 are both comprised of a polyurethane, and the distal lead body 70 is comprised of a stiffer polyurethane than the proximal lead body 72. In yet another alternative embodiment, the distal lead body 70 and the proximal lead body 72 are both comprised of a silicone, and the distal lead body 70 is comprised of a stiffer silicone than the proximal lead body 72.

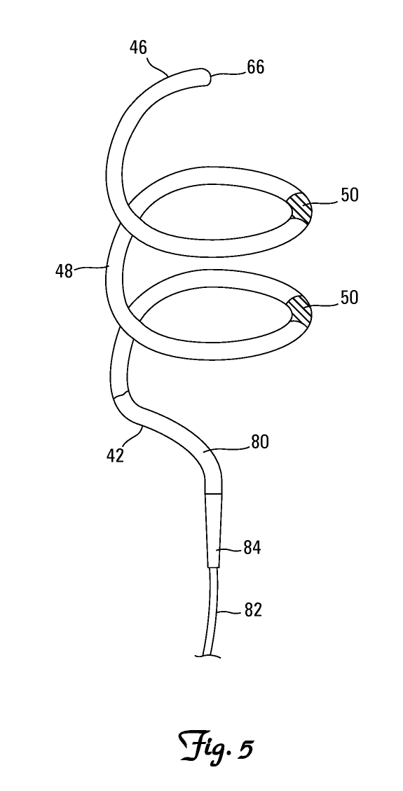

FIG. 5 shows a front view of a medical electrical lead 40 according to another embodiment of the present invention. In this embodiment, the lead body 42 comprises a distal lead body 80, a proximal lead body 82, and a transition region 84 having a stiffness that increases in a distal direction and is interposed between the distal lead body 80 and the proximal lead body 82. In the embodiment shown in FIG. 5, the increase in stiffness from the proximal region 44 to the distal region 46 is accomplished by the use of a distal lead body 80 having a thicker insulative layer (not shown) than the proximal lead body 82. In one embodiment, the thickness of the insulative layer for the proximal lead body 82 is between approximately 0.004 and approximately 0.008 inch and the thickness of the insulative layer for the distal lead body 80 is between approximately 0.006 and approximately 0.012 inch. In the embodiment illustrated in FIG. 5, the thickness of the transition region 84 varies continuously in the distal direction, but in other embodiments, the transition region 84 could comprise discrete segments having different thicknesses.

In one embodiment, the transition region 84 has a length between approximately 5 millimeters to 5 centimeters. In another embodiment, the transition region 84 has three segments of approximately equal length. Although the transition region 84 has been described with respect to the thicknesses of the insulative layer, in other embodiments, the transition region 84 could be accomplished by varying the material used to form the transition region 84. For example, in one embodiment, the transition region 84 could comprise a material that is stiffer than the material used to form the proximal lead body 82 and more flexible than the material used to form the distal lead body 80.

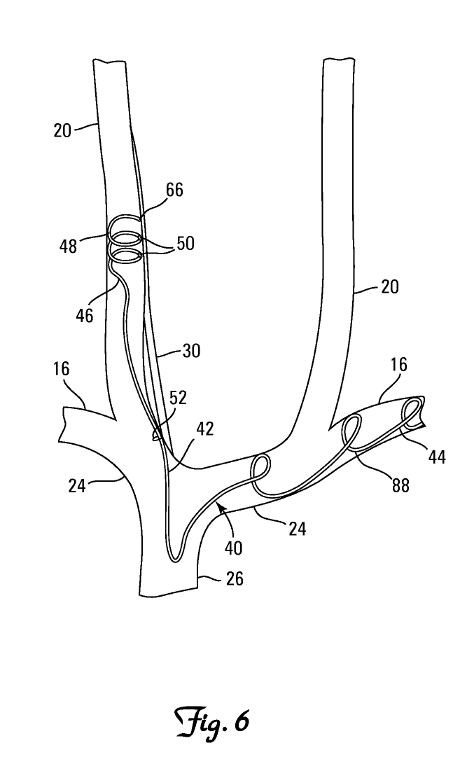

FIG. 6 shows a schematic view of a medical electrical lead 40 implanted in a patient's internal jugular vein 20 according to yet another embodiment of the present invention. In the illustrated embodiment, the medical electrical lead 40 includes a retaining structure 48 located at the distal region 46 and a formed shape 88 located at the proximal region 44. The formed shape 88, shown as a spiral in FIG. 6, acts as a weak spring to reduce the amount of force transferred from the proximal region 44 to the distal region 46, or to dampen or decouple a force or torque applied to the proximal region 44. This force reduction aspect of the formed shape 88 improves retention of the distal region 46 in the internal jugular vein 20. Although the formed shape 88 is shown as a spiral in FIG. 6, in other embodiments the formed shape 88 has any other shape that reduces the amount of force transferred to the distal region 46. In one embodiment, the formed shape 88 has the shape of a two-dimensional wave or sine curve.

The retaining structure and formed shape 48, 88 can be formed using molded silicone parts, metal conductor coils, heat formed polyurethane tubing, or any other method known in the art. The retaining structure and formed shape 48, 88 can have a variety of cross-sectional shapes, including circular or oval. In one embodiment, the retaining structure and formed shape 48, 88 comprise spirals having a pitch of between approximately zero and 5 centimeters. In an alternative embodiment, the retaining structure and formed shape 48, 88 can comprise spirals having diameters between approximately 5 and approximately 50 millimeters. In one embodiment, the retaining structure and formed shape 48, 88 have a diameter between approximately 10 and approximately 35 millimeters. In another alternative embodiment, the retaining structure and formed shape 48, 88 can have lengths when straightened from between approximately 30 and approximately 200 millimeters. In one embodiment, the lengths of the retaining structure and formed shape 48, 88 when straightened are between approximately 40 and approximately 70 centimeters.

In other embodiments, the difference in stiffness between the distal region 46 and the proximal region 44 can be accomplished through any combination of the above-disclosed embodiments. For example, in one embodiment the distal region 46 has a coil conductor 60 and a thicker distal lead body 80 while the proximal region 44 has a cable conductor 62 and a thinner lead body 82. In another embodiment, the distal region 46 has a thicker lead body 80 manufactured from a stiffer polymer while the proximal region 44 has a thinner lead body 82 manufactured from a more flexible polymer and has a formed shape 88. A transition region 84 can be used in conjunction with any combination of the above-disclosed embodiments, for example, with a coil conductor 60 and a cable conductor 62. The medical electrical lead 40 could be implanted in any vessel, such as a vein, artery, lymphatic duct, bile duct, for the purposes of nerve or muscle stimulation. The medical electrical lead 40 can include any number of conductors, electrodes, terminal connectors, and insulators.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical electrical lead for implantation in a patient's internal jugular vein at a target location adjacent to a vagus nerve, the lead comprising:
a proximal region having a proximal stiffness;
a distal region having a distal stiffness and a first spiral configured to retain the distal region in the internal jugular vein;
a transition region interposed between the proximal and distal regions and having a transitional stiffness; and
an electrode coupled to the distal region;
wherein the proximal stiffness is less than the distal stiffness so as to reduce an amount of force transferred from the proximal region to the distal region and the transitional stiffness is less than the distal stiffness and greater than the proximal stiffness.

2. The lead of claim 1 wherein the first spiral has a diameter of between approximately 5 and approximately 50 millimeters, a length of between approximately 30 and approximately 200 millimeters, and a pitch of between approximately 0 and approximately 5 centimeters.

3. The lead of claim 1 wherein a ratio of the distal stiffness to the proximal stiffness is approximately 2:1.

4. The lead of claim 1 wherein a ratio of the distal stiffness to the proximal stiffness is approximately 4:1.

5. The lead of claim 1 wherein the proximal stiffness is such that the force required for deflection of 0.5 mm over a 10 mm span of the proximal region is less than approximately 500 mN.

6. The lead of claim 1 wherein the proximal stiffness is such that the force required for deflection of 0.5 mm over a 10 mm span of the proximal region is less than approximately 300 mN.

7. The lead of claim 1 wherein the proximal region includes a formed shape.

8. The lead of claim 7 wherein the formed shape comprises a second spiral.

9. A medical electrical lead for implantation in a patient's internal jugular vein at a target location adjacent a vagus nerve, the lead comprising:
a proximal region, a distal region, and an electrode coupled to the distal region;
wherein the proximal region includes means for reducing an amount of force transferred from the proximal region to the distal region and the distal region includes means for retaining the distal region in the internal jugular vein.

10. The lead of claim 9 wherein the means for retaining comprises a retaining structure and a distal stiffness.

11. The lead of claim 10 wherein the means for reducing comprises a proximal stiffness less than the distal stiffness.

12. The lead of claim 11 wherein the means for reducing further comprises a formed shape.

13. The lead of claim 10 wherein the retaining structure comprises a spiral.

14. The lead of claim 9 wherein the means for reducing comprises a formed shape.

15. A medical electrical lead for implantation in a patient's internal jugular vein at a target location and adjacent a vagus nerve, the lead comprising:
a proximal region having a proximal stiffness;
a distal region having a distal stiffness and a retaining structure configured to retain the distal region in the internal jugular vein; and
an electrode coupled to the distal region;
wherein the proximal stiffness is less than the distal stiffness.

16. The lead of claim 15 further comprising a distal coil conductor having a distal stiffness and a proximal cable conductor having a proximal stiffness less than the distal coil conductor stiffness.

17. The lead of claim 15 further comprising a distal coil conductor having a distal stiffness and a proximal coil conductor having a proximal stiffness less than the distal coil conductor stiffness.

18. The lead of claim 15 wherein the lead further comprises a distal lead body comprising a polymer having a distal stiffness and a proximal lead body comprising a polymer having a proximal stiffness less than the distal lead body polymer stiffness.

19. The lead of claim 18 wherein the distal lead body comprises a polyurethane and the proximal lead body comprises a silicone.

20. The lead of claim 15 wherein the lead further comprises a distal lead body having a distal insulative thickness and a proximal lead body having an insulative thickness less than the distal lead body insulative thickness.
